(19) United States
(12) Patent Application Publication
(10) Pub. No.: US 2021/0007637 A1
(43) Pub. Date: Jan. 14, 2021

(54) MUSCLE NANOSENSOR FOR MINIMALLY-INVASIVE TISSUE MEASUREMENT OF MITOCHONDRIAL FUNCTIONS

(71) Applicants: THE CHILDREN'S HOSPITAL OF PHILADELPHIA, PHILADELPHIA, PA (US); THE TRUSTEES OF THE UNIVERSITY OF PENNSYLVANIA, Philadelphia, PA (US)

(72) Inventors: Zarazuela ZOLKIPLI-CUNNINGHAM, PHILADELPHIA, PA (US); Marni J. FALK, Voorhees, NJ (US); Mark ALLEN, Philadelphia, PA (US)

(73) Assignees: THE CHILDREN'S HOSPITAL OF PHILADELPHIA, PHILADELPHIA, PA (US); THE TRUSTEES OF THE UNIVERSITY OF PENNSYLVANIA, Philadelphia, PA (US)

(21) Appl. No.: 16/979,943

(22) PCT Filed: Mar. 12, 2019
(86) PCT No.: PCT/US2019/021809

§ 371 Date: Sep. 11, 2020

Related U.S. Application Data
(60) Provisional application No. 62/641,846, filed on Mar. 12, 2018.

(51) Int. Cl. A61B 5/1473; A61B 5/145; A61B 5/15; A61B 5/00

(52) U.S. Cl. CPC A61B 5/1473; A61B 5/14546; A61B 5/14542; A61B 5/150984; A61B 2505/09; A61B 2562/028; A61B 2562/0285; A61B 2562/0215; A61B 2562/0217; A61B 5/4519

(57) ABSTRACT

The present disclosure provides methods, nanosensor devices, and uses for in vivo tissue measurement of mitochondrial physiology, including tissue oxygen and other readouts, such as in mitochondrial myopathy, disease, diagnosis, biomarker assessment, and monitoring of interventions and therapies.

### PRIORITY CLAIM

This application claims benefit of priority to U.S. Provisional Application Ser. No. 62/641,846, filed Mar. 12, 2018, the entire contents of which are hereby incorporated by reference.

### BACKGROUND

### 1. Technical Field

The present disclosure relates generally to the fields of medicine, diagnostics, medical devices and nanotechnology. In certain aspects, the disclosure concerns the development and use of nanosensor devices for in vivo tissue measurement of mitochondrial physiology, including but not limited to O₂ levels, and use thereof.

### 2. Background

Mitochondrial disease is a clinically heterogeneous group of >350 gene disorders that collectively affect at least 1 in 4,300 people across all ages [1]. Myopathy is the most frequent finding [2], for which no FDA-approved therapies exist [3, 4]. Mitochondrial myopathy refers to exercise intolerance and muscle weakness caused by mitochondrial dysfunction due to mutations in either nuclear or mitochondrial genes. A systematic review identified only 12 methodologically robust mitochondrial disease clinical trials conducted over the past two decades, bearing no efficacious evidence [4]. A major contributing factor to the scarcity of randomized clinical trials (RCTs) is the absence of validated biomarkers or outcome measures that correlate with disease severity, progression, and response to intervention [5-7]. Non-MM specific motor performance measures exist but none have been validated in MM; still, these are being used as outcome measures in MM clinical trials [8, 9]. The increasing pursuit of MM treatment trials has created a pressing need for robust natural history studies and quantitative outcome measures that reliably reflect MM disease severity, progression, and therapeutic response. Since effective therapies may only incrementally slow disease progression, quantitative outcome measures are needed that are specific to MM, and highly sensitive to clinical or biochemical changes. Currently, no methods to directly quantify muscle tissue oxygen (O₂) levels to assess in vivo muscle oxidative phosphorylation (OXPHOS) capacity exist.

There is limited understanding of the natural history of all mitochondrial diseases. Novel nuclear and mitochondrial gene defects are increasingly identified [10], with new clinical phenotypes emerging that were not previously appreciated. While a few existing longitudinal studies have been done in adults with defined clinical syndromes (e.g., Chronic Progressive External Ophthalmoplegia) [10], none exist for genetically and/or biochemically defined MM.

Yet, 2 recent phase II pharmaceutical trials were conducted in MM. An accurate understanding of MM natural history throughout the lifespan is needed to be able to demonstrate clinically meaningful intervention effects and facilitate new outcome measures development. The ability to conduct longitudinal assessments of muscle O₂ as a marker of OXPHOS in an individual with MM over time, including periods of acute illness or surgical interventions, would provide great depth of understanding of natural history.

The inherent complexity of MM and poorly defined phenotypes leads to challenges in diagnosis and effective disease monitoring. Historically, an open biopsy to obtain muscle for ex vivo spectrophotometric-based electron transport chain enzyme activity assays [11] has been the gold standard approach to measure muscle OXPHOS capacity [12]. While this approach can provide a highly precise definition of mitochondrial function, it is suboptimal for several reasons. First, it is invasive and costly, prohibiting its use in routine screening, disease monitoring, and clinical trials. Further, results are not accessible in real-time, inadequate specimen handling influences results, and biopsies are performed typically only once in a patient's lifetime as general anesthesia is often poorly tolerated. In addition, novel molecular mechanisms such as mtDNA maintenance defects may occur that are not discerned by enzymatic testing. Lastly, in the current molecular diagnostic era, a muscle biopsy is no longer the first diagnostic step [13]. However, selective testing tissue can still be highly informative, particularly for mitochondrial DNA (mtDNA)-based disease. Overall, there exists a critical need to harness repeatable, less invasive, and quantitative methods of OXPHOS dysfunction in MM.

Mitochondrial dysfunction may also be present in a range of disorders without frank myopathy. Having a way to test muscle or other tissue in vivo mitochondrial capacity at baseline and with stressors such as exercise would be highly useful to detect and optimize mitochondrial dysfunction for diagnostic, biomarker, exercise training, acute resuscitation, and/or therapeutic monitoring purposes in a range of medical and non-medical applications.

### SUMMARY

Thus, in accordance with the present disclosure, there is provided an implantable oxygen (O₂) nanosensor comprising:

(a) an O₂ sensor comprising:
(i) a working electrode;
(ii) a reference electrode;
(iii) a counter electrode;
(iv) an inner electrolyte cell; and
(v) an O₂-permeable membrane bounding at least one portion of the inner electrolyte cell,

and optionally

(b) nanofiber material surrounding the O₂ sensor.

The nanosensor may be a Clark-type O₂ sensor comprising:
(i) a working electrode;
(ii) a reference electrode;
(iii) a counter electrode;
(iv) an inner electrolyte cell;
(v) reaction chamber; and
(vi) an O₂-permeable membrane separating the inner electrolyte cell and the reaction chamber,
   The nanofiber material may be in the form of a nanofiber mesh tube, such as a polycaprolactone nanofiber mesh tube. The nanosensor may further comprise a voltage source and/or an ammeter. The nanosensor may be about 2.5 mm in diameter, or about 1.8 mm in diameter, or between 1.0 and 1.8 mm in diameter, or smaller, such as between 0.1M and 0.5 mm. The working electrode may be a platinum electrode and/or the counter electrode is a silver electrode. The O₂-permeable membrane may be a Teflon membrane. The O₂-permeable membrane may be a permselective membrane. The nanosensor may comprise materials that are biodegradable. The nanosensor may comprise of wires that are biodegradable. The nanosensor may comprise of a sensor that is attached by wires to a detector for analysis. The nanosensor may comprise of remote analysis without wire attachment. The nanosensor may comprise of a sensor embedded in a transdermal puncture device. The nanosensor may comprise of a device inserted from an external puncture device or needle microarray. The nanosensor may detect O₂ levels alone and/or in combination with one or more of the following physiologic read-outs including but not limited to: calcium, potassium, sodium, chloride, pH, bicarbonate, carbon dioxide, hydrogen peroxide, temperature, lactate, pyruvate, nicotinamide adenine dinucleotide (NADH or NAD+), adenosine triphosphate (ATP), adenosine diphosphate (ADP), adenosine monophosphate (AMP), ammonia, acetoacetate, beta hydroxybutyrate, or emitted light.

In another embodiment, there is provided a method of measuring oxygen (O₂) level in a tissue in vivo comprising (i) implanting the implantable O₂ nanosensor of claim 1 into a muscle tissue of a subject, and (ii) assessing O₂ level as a function of the conversion of O₂ into H₂O and resulting current generated. The subject may be a non-human animal including but not limited to mouse, rat, pig, other mammal, zebrafish (Danio rerio), worm (Caenorhabditis elegans), or a human. Insertion may be temporary or permanent, and device used may be percutaneous/transdermal or intramuscular full insertion or implant. Implanting may be into any muscle such as the subjects forearm muscle, thigh or gluteal muscle, or any muscle including eye, eyelid, facial, arm, leg, hand, foot or trunk. Implanting may also comprise into other tissues including the subject's brain, heart, liver, intestines, pancreas, urinary bladder, uterus, kidney, adrenal gland, thyroid gland, bone, cartilage, joint, or eye.

Implanting may also comprise inserting the O₂ nanosensor into a needle or needle array, introducing the needle or needle array into the any tissue such as muscle, and deploying the O₂ nanosensor into the muscle tissue. The method may also comprise after step (i), and before step (ii), subjecting the subject to physical exercise, including anything from mild, moderate to severe physical exercise to physical exhaustion. The method may also comprise subject that are acutely ill, such as but not exclusively from trauma, surgery, infection, sepsis, stroke, heart attack, hemorrhage or shock. The subject may be suspected of or diagnosed as suffering from a disease or disorder, such as mitochondrial myopathy, primary mitochondrial disease, secondary mitochondrial disease, mitochondrial dysfunction, or no known disease in whom mitochondrial function is being monitored and optimized in states of severe health or acute or chronic disease. The method may also comprise subjects being medically monitored for resuscitation purposes or being evaluated on the sports field or for medical purposes for post-concussion mitochondrial pathology. The method may comprise a subject in the general population seeking to optimize their exercise training regimen. The method may comprise a subject is in the general population seeking to optimize their nutrition regimen. The method may comprise a subject in the general population seeking to evaluate mitochondrial effects of their medication. The method may comprise a subject in the general population seeking to evaluate mitochondrial effects of lifestyle choices, including but not limited to diet, medications, exercise, drug use, tobacco exposure, environmental toxin exposure, or chemical exposure. The method may comprise a subject who is an athlete or participating in athletic training. The method may comprise who is a military recruit or member. The method may comprise a family member with mitochondrial disease or dysfunction. The method may comprise a primary or secondary mitochondrial disorder without known myopathic features. The method may comprise a disease or disorder involving secondary mitochondrial dysfunction. The method may comprise a subject being evaluated for disease prognosis or progression. The method may comprise a subject who is being evaluated for therapeutic response to a candidate therapy, therapies, or therapeutic intervention. The method may consist of purposes that include but are not limited to safety assessments of exercise performance, capacity, and training; safety assessments in post-concussion; safety assessments in military training or injuries; diagnosis of primary (genetic based) mitochondrial disease; diagnosis of secondary mitochondrial disease; monitoring in vivo mitochondrial respiratory capacity; predicting and assessing organ failure at a pre-critical or critical level; as a clinical trial outcome measure to assess mitochondrial disease or dysfunction natural history and progression; as a clinical trial outcome measure to assess mitochondrial disease response to a candidate therapy, therapies, or therapeutic intervention; as a clinical diagnostic test for mitochondrial dysfunction; as a clinical diagnostic test for mitochondrial disease severity, progression, and therapeutic response.

Other objects, features and advantages of the present disclosure will become apparent from the following detailed description. It should be understood, however, that the detailed description and the specific examples, while indicating particular embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request.

FIGS. 1A-C—Results of ergometry. (FIG. 1A) Peak muscle O₂ consumption (VO2 max, mls/kg/min), (FIG. 1B) Peak work rate (W/kg) and (FIG. 1C) Anaerobic threshold (AT, mls/kg/min) results were measured in ‘Definite’ (n=15), ‘Probable’ (n=10), ‘Possible’ (n=12) and ‘Unlikely’ (n=26) MM subjects, as compared to other genetic diagnoses (n=3), cardiomyopathy from other etiologies (n=52), and healthy subjects (n=97). VO₂ₘₐₓ, AT and Peak work in ‘Definite’ or ‘Probable’ MM subjects was significantly lower than in cardiomyopathy (n=52) or healthy control groups (n=97) and trended lower than in ‘Possible’ or ‘Unlikely’ MM subjects. *p<0.05, **p<0.01, ***p<0.001, ***p<0.0001. In summary, these results indicate that the subjects with Definite MM were distinctly low in their muscle aerobic and physical capacity, indicating that bicycle ergometry is highly sensitive to distinguish MM from deconditioned ‘unlikely’ MM and control subjects. However, ergometry was technically challenging for individuals with imbalance and in young children. These limitations support the rationale to measure in vivo muscle O₂ levels with an O₂ nanosensor, which would not be limited in its utility by age or physical limitations.

FIG. 2—Comparison of distance walked at 1 and 6 minutes in MM subjects. The 6 MWT is an objective evaluation of exercise tolerance. These results indicate that MM subjects walked 13.8 meters less in the 6th minute compared to the 1st minute (n=11). This novel analytic approach comparing last to initial minute times (rather than cumulative time) may provide a more sensitive measure of fatigue in MM. compared to total distance walked alone. In summary, these results confirm that Definite MM patients display fatigue, as evidenced by significantly shorter distance walked in the 6th minute. The pathophysiology of fatigue is not understood in primary mitochondrial disease, and better understanding may lead to specific treatment interventions. This supports the need for the capabilities of the O₂ nanosensor that is able to provide a direct, objective measure of muscle mitochondrial function in vivo during physical performance.

FIG. 3A—Muscle O₂ levels in C57BL6J control male mice compared to mt-ND6 mouse model of MM. In C57BL6J control mice, sedentary (n=8) and treadmill exercised mice (n=8) revealed similar muscle O₂ levels of 33.9±3.4 and 39.1±4.0 Torr (mean±SEM), respectively. In C57BL6J mice carrying a mitochondrial complex 1-ND6 mutation (m.13885C), sedentary (n=5) and treadmill-exercised (n=3) mice had muscle O₂ levels of 37.5±2.8 Torr versus 73.5±0.68 (mean±SEM), respectively. This increased tissue O₂ level after exercise supports the hypothesis that exercise is associated with a further increase in muscle O₂ levels in MM.

FIG. 3B—Measurement of muscle O₂ levels in healthy control zebrafish revealed similar muscle O₂ levels of 33.7±4.3 Torr and 32.1±0.38 Torr in sedentary (n=3) and exercised fish (n=3), respectively.

FIG. 4A—Image of the first-generation prototype O₂ nanosensor, diameter size of 2.4 mm. FIG. 4B—Image of the second-generation 1.8 mm diameter prototype O₂ nanosensor, compared to the larger first-generation prototype.

FIG. 5A—Image of wired-O₂ nanosensor in mouse muscle.

FIG. 5B—Image of wired-O₂ nanosensor in zebrafish muscle.

### DESCRIPTION OF ILLUSTRATIVE EMBODIMENTS

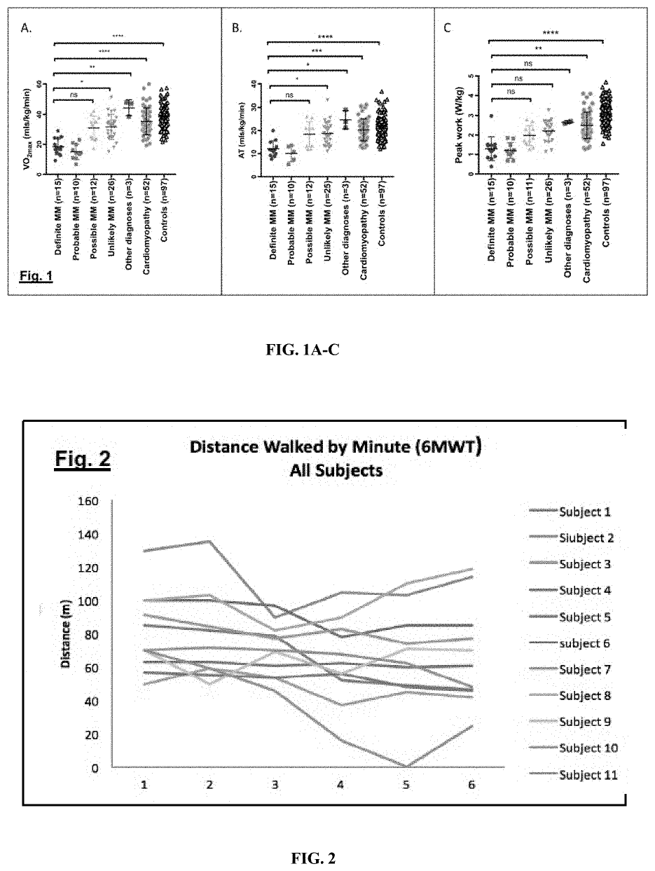

Implantable nanosensors are an emerging class of devices with the ability to measure a particular analyte in vivo, which may potentially redefine understanding of a disease by providing new insights into disease mechanisms. Whole body, blood, and tissue O₂ levels are elevated in MM due to impaired O₂ extraction efficiency when mitochondrial oxidative phosphorylation (OXPHOS) function is compromised [14, 15]. An in vivo nanosensor will provide real-time measurements of OXPHOS, for which the implications could be substantial given known fluctuations in disease stability. This also has utility to evaluate mitochondrial function without having frank myopathy. As no methods currently exist for direct measurement of muscle mitochondrial function in vivo, the authors have studied various clinical assessments, as indicated in FIG. 1A-C and FIG. 2. However, the utility of these clinical approaches is limited by age, height, ability to follow instructions and necessitates the need to be in the hospital laboratory at the time the measurements are obtained, hence do not facilitate measurements in real time during normal daily activities, exercise training or acute illness when the subject is at home or not in hospital.

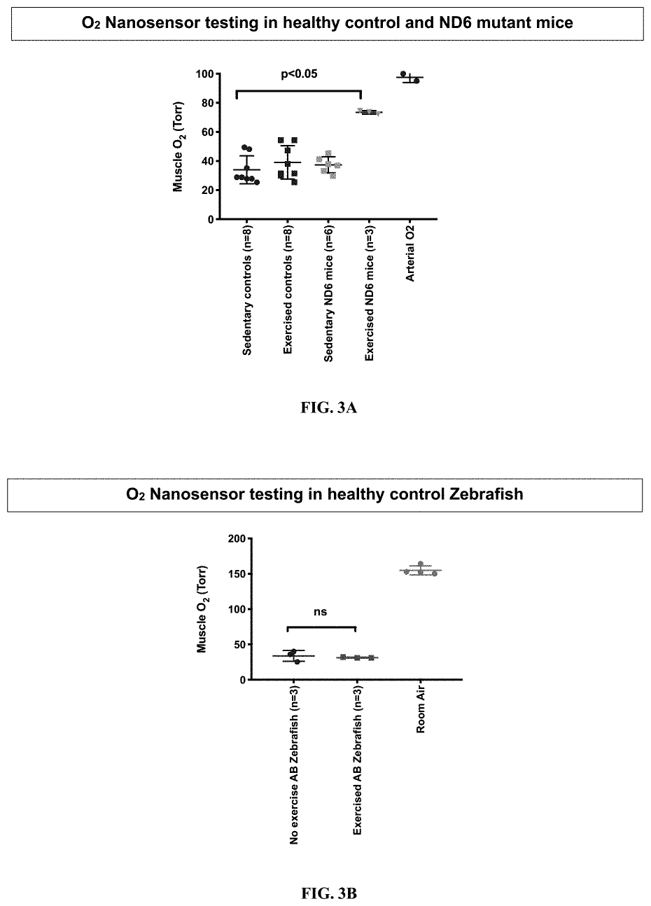

Next-generation nanotechnologies are now readily accessible that will support minimally-invasive, repeated, quantitative assessments of mitochondrial function in patients. Using such, the inventors have fabricated a clinical prototype electrochemical (e.g. Clark-type) O₂ Nanosensor and tested it in MM Zebrafish and mouse models (see Examples) and stainless steel (SS)-needle microarrays shown to be safe in animals [16]. In an in vivo gluteal muscle study, MCAT (Mitochondrial Catalase) transgenic mice that overexpress the mitochondrial antioxidant, Catalase, and therefore with normal OXPHOS capacity, were measured for muscle O₂ and found to exhibit 53.4±3.58 Torr (n=2), similar to a prior report of muscle O₂ levels where muscle O₂ was measured using a immunochemical detection utilizing immunofluorescence [17]. In preliminary studies in the mt-ND6 complex I mutant MM mice, muscle O₂ was 37.5±2.8 Torr when sedentary (n=5) and 73.5±0.68 Torr following treadmill-exercise (n=3) (FIG. 3A). These data demonstrate the expected increase in tissue O₂ levels in MM mice, with further increase upon exercise. Measurement of muscle O₂ levels in control zebrafish revealed similar muscle O₂ levels of 33.7±4.3 Torr and 32.1±0.38 Torr in sedentary (n=3) and exercised fish (n=3), respectively (FIG. 3B). Additional studies in all MM mouse and zebrafish models of mitochondria' disease and healthy control animals are underway.

Moreover, an institutional review board protocol for first in-human testing in MM subjects is underway. During exercise, continuous measurements can be obtained at a constant workload to obtain insight into the work capacity of the mitochondria. Future plans include modifying, optimizing, and testing in human MM subjects an O₂ nanosensor-microarray to measure intramuscular O₂ levels in vivo, permitting minimally-invasive measure of mitochondrial OXPHOS capacity. Additional nanosensor readouts of mitochondrial function that will be supplemented to tissue O2 measurement include but are not limited to calcium, potassium, sodium, chloride, pH, bicarbonate, carbon dioxide, hydrogen peroxide, temperature, lactate, pyruvate, nicotinamide adenine dinucleotide (NADH or NAD+), adenosine triphosphate (ATP), adenosine diphosphate (ADP), adenosine monophosphate (AMP), ammonia, acetoacetate, beta hydroxybutyrate, or emitted light.

These and other aspects of the disclosure are set out in detail below.

### I. DEFINITIONS

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and“included”, is not limiting. Also, terms such as “element” or “component” encompass both elements and components comprising one unit and elements or components that comprise more than one unit unless specifically stated otherwise.

As used herein, the term “about,” when used in conjunction with a percentage or other numerical amount, means plus or minus 10% of that percentage or other numerical amount. For example, the term “about 80%,” would encompass 80% plus or minus 8%.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

As used herein, and unless otherwise indicated, the terms “disease”, “disorder” or “condition” refer to a state of being or health status of a patient or subject capable of being treated with a compound, pharmaceutical composition, exercise, lifestyle change, or method provided herein.

As used herein, and unless otherwise indicated, the terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In some embodiments, “treating” refers to the treatment of cancer.

As used herein, and unless otherwise indicated, the terms “prevent,” “preventing,” and “prevention” contemplate an action that occurs before a patient begins to suffer from a disorder that involves cancer that delays the onset of, and/or inhibits or reduces the severity of cancer.

As used herein, and unless otherwise indicated, the terms “manage,” “managing,” and “management” encompass preventing, delaying, or reducing the severity of a recurrence of a disorder such as cancer in a patient who has already suffered from such a disease, disorder or condition. The terms encompass modulating the threshold, development, and/or duration of the disorder or changing how a patient responds to the disorder.

As used herein, and unless otherwise specified, a “therapeutically effective amount” of a compound is an amount sufficient to provide any therapeutic benefit in the treatment or management of a disorder. A therapeutically effective amount of a compound means an amount of the compound, alone or in combination with one or more other therapies and/or therapeutic agents that provide any therapeutic benefit in the treatment or management of a disorder.

As used herein, and unless otherwise specified, an “effective amount” is an amount sufficient for a compound to accomplish a stated purpose relative to the absence of the compound (e.g. achieve the effect for which it is administered). An example of a “therapeutically effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, “patient” or “subject in need thereof” refers to a living organism suffering from or prone to a disease or condition that can be treated by administration of a composition or pharmaceutical composition as provided herein. A subject may also refer to an individual in the general population, athlete, military member, etc., in whom mitochondrial function is being monitored for purposes of improved understanding and optimization of their mitochondrial function and capacity in states of health and disease. Non-limiting examples include humans, primates, companion animals (dogs, cats, etc.), other mammals, such as but not limited to, bovines, rats, mice, monkeys, goat, sheep, cows, deer, as well as other non-mammalian animals including vertebrates (such as zebrafish, D. rerio) and invertebrates (worms, C. elegans). In some embodiments, a patient is human.

### II. MITOCHONDRIAL DISEASE

A. Mitochondrial Myopathy

Mitochondrial myopathies are types of myopathies associated with mitochondrial disease. On biopsy, the muscle tissue of patients with these diseases usually demonstrate “ragged red” muscle fibers. These ragged-red fibers contain mild accumulations of mitochondrial glycogen and neutral lipids and may show an increased reactivity for succinate dehydrogenase and a decreased reactivity for cytochrome c oxidase. Inheritance was believed to be maternal (non-Mendelian extranuclear). It is now known that certain nuclear DNA deletions can also cause mitochondrial myopathy such as but not limited to the OPA1 gene mutation. There are several subcategories of mitochondrial myopathies.

Signs and symptoms include (for each of the following causes):
Mitochondrial encephalomyopathy, lactic acidosis, and stroke-like syndrome (MELAS)
   Varying degrees of cognitive impairment and dementia
   Lactic acidosis
   Strokes
   Transient ischemic attacks
   Hearing loss
   Weight loss
Myoclonic epilepsy and ragged-red fibers (MERRF)
   Progressive myoclonic epilepsy
   Clumps of diseased mitochondria accumulate in muscle fibers and appear as “ragged-red fibers” when muscle is stained with modified Gomori trichrome stain
   Short stature
Kearns-Sayre syndrome (KSS)
   External ophthalmoplegia
   Cardiac conduction defects
   Sensorineural hearing loss
Chronic progressive external ophthalmoplegia (CPEO)
   Progressive ophthalmoparesis
   Symptomatic overlap with other mitochondrial myopathies
      Although no cure currently exists, there is hope in treatment for this class of hereditary diseases with the use of an embryonic mitochondrial transplant. There are a wide range of other multi-system mitochondrial diseases, in which overt myopathy may or may not be a feature, but that tissue-level physiology assessments may be a useful diagnostic, prognostic, or therapeutic response measure for many disorders beyond classic MM.

B. Study of MM

Myopathy is the lead symptom in MM. In a prospective survey the inventors performed of 290 adults and children self-reported to have mitochondrial disease, subjects reported a mean of 15.6+5.9 (S.D.) symptoms. Muscle weakness (95.4%), fatigue (95.1%) and exercise intolerance (94.7%) were the most commonly experienced and major motivating symptoms for clinical trial participation [2]. The results of this study help inform mitochondrial clinical trial design to ensure the symptoms that matters most to patients are targeted. Fatigue and exercise intolerance often have the greatest impact on MM and mitochondrial disease daily life [18]. While muscle weakness is the predominant symptom in Duchenne Muscular Dystrophy (DMD), exercise intolerance and fatigue often exist in MM despite an absence of overt muscle weakness. Therefore, a clinical assessment that focuses solely on muscle strength will not reliably reflect the severity of mitochondrial dysfunction that underlies MM or the most common symptoms of mitochondrial disease.

Recent MM clinical trials to date have only been conducted in adult subjects. The natural history and treatment response is not likely similar in MM children and adults. In general, the earlier the MM onset, the more severe the disease course. Therefore, MM children are an ideal cohort to recruit to clinical trials if they can be readily evaluated. To successfully treat all MM individuals, one must understand the natural history and utility of specific outcome measures across the age span. For many muscle diseases, such as Duchenne Muscular Dystrophy, assessments of limb strength are a crucial outcome measure. In MM clinical trials, measures of overall motor abilities may be more useful, particularly timed measures of gait (FIG. 2), noting that weakness, ataxia and spasticity may complicate interpretation.

### III. DEVICE

A. O₂ Sensor

Exemplary embodiments include an O₂ sensor, including for example, an electrochemical O₂ sensor. In particular embodiments, the electrochemical O₂ sensor may be configured as a Clark cell, also referred to as a Clark-type O₂ sensor or Clark electrode. The Clark electrode is an electrode that measures ambient oxygen concentration in a liquid using a catalytic platinum surface according to the net reaction:

O₂+4 e⁻+4 H⁺→2 H₂O

It improves on a bare platinum electrode by use of a membrane to reduce fouling and metal plating onto the platinum. Leland Clark had developed the first bubble oxygenator for use in cardiac surgery. However, when he came to publish his results, his article was refused by the editor since the oxygen tension in the blood coming out from the device could not be measured. This motivated Clark to develop the oxygen electrode.

The electrode, when implanted in vivo, would reduce oxygen and thus required stirring in order to maintain an equilibrium with the environment. Severinghaus improved the design by adding a stirred cuvette in a thermostat. A discrepancy between the measured partial pressure of oxygen (pO₂) between blood samples and gaseous mixtures of identical pO₂, meant that the modified electrode required calibration; consequently, a microtonometer was added to the water thermostat.

The electrode compartment is isolated from the reaction chamber by a thin Teflon membrane; the membrane is permeable to molecular oxygen and allows this gas to reach the cathode, where it is electrolytically reduced.

The above reaction requires a steady stream of electrons to the cathode, which depends on the rate at which oxygen can reach the electrode surface. Increasing the voltage applied (between the platinum (Pt) electrode and a second silver (Ag) electrode) will increase the rate of electrocatalysis. Clark affixed an oxygen permselective membrane over the Pt electrode. This limits the diffusion rate of oxygen to the Pt electrode.

Above a certain voltage, the current plateaus and increasing the potential any further does not result in a higher rate of electrocatalysis of the reaction. At this point, the reaction is diffusion-limited and depends only on the permeability properties of the membrane (which is ideally well characterized, the electrode being calibrated against known standard solutions) and by the oxygen gas concentration, which is the measured quantity.

The Clark oxygen electrode laid the basis for the first glucose biosensor (in fact the first biosensor of any type), invented by Clark and Lyons in 1962. This sensor used a single Clark oxygen electrode coupled with a counter-electrode. As with the Clark electrode, a permselective membrane covers the Pt electrode. Now, however, the membrane is impregnated with immobilized glucose oxidase (GOx). The GOx will consume some of the oxygen as it diffuses towards the PT electrode, incorporating it into H₂O₂ and gluconic acid. The rate of reaction current is limited by the diffusion of both glucose and oxygen. This diffusion can be well characterized for a membrane for both the oxygen and glucose, leaving as the only variable the oxygen and glucose concentrations on the analyte-side of the glucose membrane, which is the quantity being measured.

B. Device of the Present Disclosure

The inventors have custom designed, fabricated and characterized a prototype O₂ sensor in tissue-engineered polycaprolactone (PCL) nanofiber mesh tube that measures 2.4 mm in diameter, and a second-generation prototype that measures 1.8 mm in diameter. In certain embodiments, the O₂ sensor may be configured as an electrochemical sensor, and in specific embodiments, the O₂ sensor may be configured as a Clark-type sensor. Other materials may be employed to coat the sensor. Examples of natural polymers include collagen, cellulose, silk fibroin, keratin, gelatin and polysaccharides such as chitosan and alginate. Examples of synthetic polymers include poly(lactic acid) (PLA), polyurethane (PU), poly(lactic-co-glycolic acid) (PLGA), poly(3-hydroxybutyrate-co-3-hydroxyvalerate) (PHBV), and poly(ethylene-co-vinylacetate) (PEVA). Smaller size prototypes are being generated.

In one sensor's working embodiment, O₂ diffuses through the O₂-permeable membrane into the inner electrolyte cell, producing a current proportional to the amount of reduced O₂ when a negative potential is applied between the working and reference electrodes. The nanosensor is manually inserted in anesthetized muscle. It has been validated in mice gluteus muscle within 90 seconds from completion of treadmill-exercise to insertion of sensor (FIG. 3A). A range of genetic mouse models of mitochondrial myopathy have been compared to validate the device, including MM mice harboring the mt-ND6 mutations, and transgenic MCAT mice, both at baseline and following 20 minutes of standard-protocol treadmill exercise. A range of genetic zebrafish models of mitochondrial myopathy including fish harboring the SURF1 mutation have been tested, demonstrating the feasibility to validate the nanosensor in various tissues of vertebrate animal models with a range or primary mitochondrial diseases.

### IV. METHODS OF ASSESSING MUSCLE OXYGENATION

In terms of implantation, virtually any muscle group may be utilized. However, the inventors hypothesize that use of the O₂ nanosensors in small (rather than larger) muscle groups will be most technically feasible and provide clinically meaningful results. For example, one target muscle will be the forearm muscles. Additional tissues may also be readily studied, depending on desired indication, including but not limited to eye, eyelid, facial, arm, leg, hand, foot, or trunk.

Another aspect of the method is the deployment of a stiff delivery needle or needle array that can be used to successfully insert the O₂ Nanosensor and which is subsequently removed. Preliminary animal validation experiments in brain phantom material using the array showed in vivo intracortical recording capabilities of the ECM-NEs from rat motor cortex [26].

The needle microarray will be tested and customized in human healthy volunteers to guide the fabrication of microarray needle lengths required for adequate tissue perforation. Intramuscular or other tissue location of the SS needles will be confirmed by ultrasound. Once inserted, a test subject will perform an exercise regimen (e.g., forearm grip or leg cycle ergometry) to the point of exhaustion to independently define their maximal VO₂, Anaerobic Threshold (AT), and workload [27, 28]. This will promote a stimulus-response paradigm to measure mitochondrial respiratory capacity by standard methodology in comparison to the nanosensor device(s).

To test device safety and utility of the O₂ Nanosensor-needle prototype, healthy volunteers will be tested at rest and after ergometry. Specifically, all subjects will undergo placement of the device under topical anesthetic and then perform forearm grip exercises and/or leg bicycle ergometry to define maximal VO₂, AT, & workload. Forearm muscle O₂ measurements will be obtained at rest, during exercise and immediately after exercise to ascertain optimal timing of measurements, and to measure the capacity of the mitochondria to perform work. The inventors will evaluate the length of time required for accurate O₂ measurements and assess for motion artifacts. To evaluate reproducibility, all subjects will be tested twice, 7 days apart. These studies will validate the O₂ nanosensor prototype utility and sensitivity in human subjects.

O₂ Nanosensor-needle microarray measurements should not only be feasible and tolerable in all subjects but should be able to reliably demonstrate that muscle O₂ levels will be increased in MM patients compared to control subjects, at least after exercise. These muscle O₂ measurements should correlate with ergometry read-outs of OXPHOS function.

TABLE 1
Pathophysiology of MM, and contextual model of expected changes
in healthy and MM individuals at rest and after exercise.*

| | HEALTHY | | MM | |
|---|---|---|---|---|
| | Resting | Excercise | Resting | Exercise |
| Inherent mitcochondrial | Normal | Normal or ↑ | ↓ | ↓↓ |
| OXPHOS capacity | | | | |
| Mitochondrial O₂ uptake | Normal | ↑ | ↓ | ↓↓ |
| Muscle tissue O₂ levels | Normal | Normal or ↓ | ↑ | ↑↑ |

*In MM, mitochondrial OXPHOS defects give rise to symptoms of myopathy, decreased mitochondrial O₂ uptake, and subsequent increased muscle O2 and altered COX redox levels, which are exacerbated by exercise.

### V. EXAMPLES

The following examples provides further details regarding examples of various embodiments. It should be appreciated by those of skill in the art that the techniques disclosed in the examples that follow represent techniques and/or compositions discovered by the inventors to function well. However, those of skill in the art should, in light of the present disclosure, appreciate that many changes can be made in the specific embodiments which are disclosed and still obtain a like or similar result without departing from the spirit and scope of the disclosure. These examples are illustrations of the methods and systems described herein and are not intended to limit the scope of the disclosure. Non-limiting examples of such include but are not limited to those presented below.

### Example 1

Patient cohort. The CHOP Mitochondrial Medicine Frontier Program collectively evaluates patients/year, including an existing cohort of 180 individuals with genetically-confirmed, definite, primary mitochondrial disease (CHOP IRB #08-6177, PI Falk). As a site in the North American Mitochondrial Disease Consortium (Falk PI, Zolkipli, Co-PI), they can also recruit from a registry of 1,400 primary mitochondrial disease subjects. A cohort of 90 adult and pediatric MM individuals, having genetically and/or biochemically-confirmed mitochondrial disease with predominant symptoms of myopathy, are also enrolled in an active study (CHOP IRB #16-013364, PI Zolkipli). The inventors have now collected standardized data of motor assessments on these 90 MM adults and children to support new outcome measure development in a planned NIH funded project.

Clinical impact of muscle weakness, fatigue, and exercise intolerance. The inventors conducted a qualitative study of the impact of MM symptoms on daily life. Semi-structured patient or caregiver interviews were conducted in 30 MM subjects (manuscript in preparation). Qualitative analysis (NVivo) revealed common themes of fatigue, such as “Physically, mentally, and emotionally overwhelming. I don't have enough energy to function normally. Any little thing that happens, it just drains me”, and exercise intolerance, such as “Activities of daily life are like exercise. The more I do, the more time I have to spend recuperating”. These results underscore the significant impact of mitochondrial dysfunction on MM symptoms in daily activities.

Limitations of bicycle ergometry. The inventors have studied the utility of bicycle ergometry in 63 individuals evaluated in their clinical center to quantify their exercise intolerance (FIGS. 1A-C). They found that 3 parameters (aerobic capacity, VO₂ₘₐₓ; physical capacity, peak work (W/kg); anaerobic threshold, AT) are discriminatory for subjects with ‘definite’ and ‘probable’ MM, as compared to those with ‘possible’ or ‘unlikely’ MM (Zolkipli et al., in preparation). Subjects were grouped by Modified Bernier Criteria [29]. A high prevalence of physical deconditioning was present among subjects in the ‘possible’ and ‘unlikely’ MM groups. These data align well with prior observations that physical deconditioning may lead to low aerobic and physical capacities, and false positive ergometry results [19, 30], although the patients with definite MM remained distinctly low in their muscle aerobic capacity. The procedure was noted to be technically challenging for individuals with significant imbalance and could not be performed in young children. These data also show sensing mitochondrial function for purposes of optimization through nutrition or exercise has utility beyond those with primary mitochondrial disease.

Comparison of 1 to 6 minute walking distance in 6 minute walk test (6 MWT). The inventors have been exploring various analyses of 6 MWT data in MM. Previous evaluations of the 6 MWT as a measure of fatigue in MM showed poor correlation with disease symptoms [31]. While boys with DMD and healthy individuals maintain consistent walking speed throughout the 6 MWT [21], preliminary results shown in FIG. 2 show that revised timed walk tests may better characterize MM fatigue. The pathophysiology of fatigue is not understood in primary mitochondrial disease, and better understanding may lead to specific treatment interventions. This supports the need for the capabilities of the O₂ nanosensor that is able to provide a direct, objective measure of muscle mitochondrial function in vivo during physical performance.

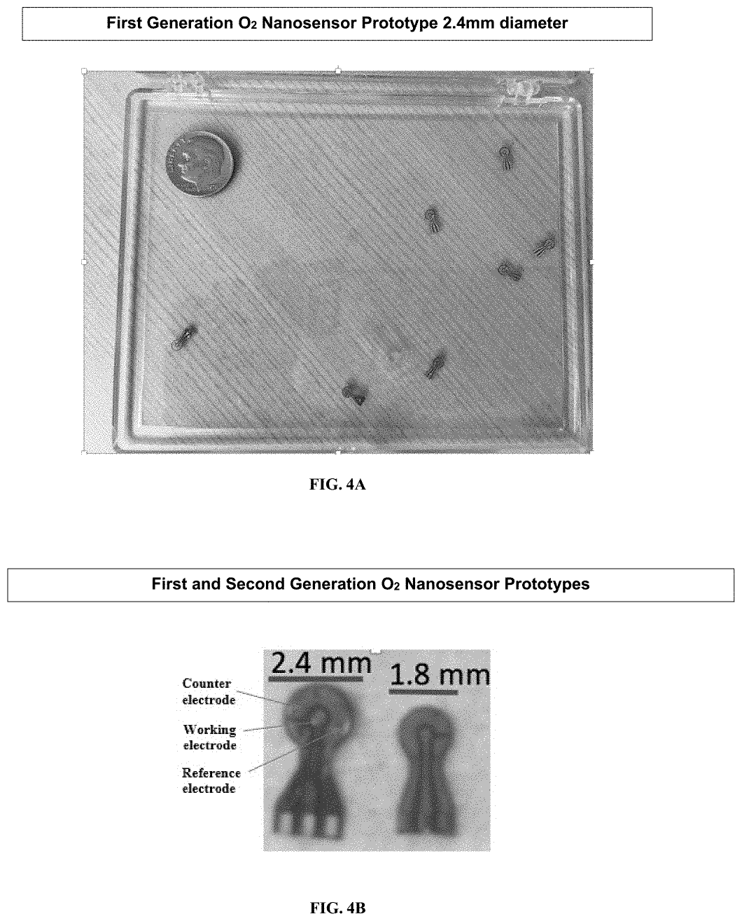

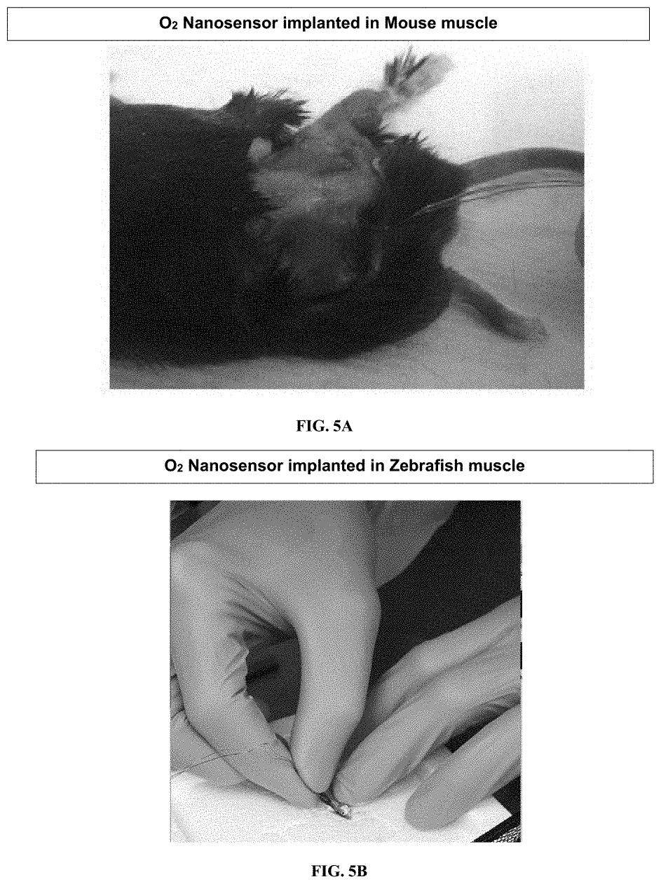

Mouse muscle O₂ measurement using O₂ Nanosensor. In certain embodiments, the electrochemical O₂ sensor features a 3-electrode configuration, including for example a Clark-type O₂ sensor. This includes working, reference, and counter electrodes (FIG. 4B), as well as an inner electrolyte cell, and O₂-permeable membrane.
(A) Electrochemical characterization of O₂ nanosensor. Benchtop electrochemical testing of the O₂ nanosensor demonstrated O₂ sensitivity of −14.4 nA/atm. % and excellent linearity, with a correlation coefficient of 0.99. Further bench-testing indicates stable sensor performance under varying temperatures (34-41° C.), pH (6.5-8.0), and on exposure to different sterilization methods including autoclave, ethylene oxide, and UV radiation (data not shown).
(B) Preclinical testing of O₂ nanosensor. The inventors have conducted testing of the O₂ sensor in C57BL/6J control and the ND6 mouse model of MM. Preliminary results in FIG. 3A confirm feasibility and efficacy of the O₂ Nanosensor to detect increased muscle O₂ after exercise in MM mouse muscle.

Summary of Preliminary Data. In summary, the inventors have shown that bicycle ergometry can detect distinctly low whole body VO₂ in MM subjects, supporting the rationale to study its utility in a natural history study. They have also found that MM subjects fatigue in the final (6th) as compared to the 1st minute on 6 MWT. The inventors are performing a natural history study of genetically-confirmed pediatric and adult MM subjects to identify clinical markers of MM disease progression. Validation of the comparative utility of the O₂ nanosensor will be readily achieved in this context.

The inventors have also now demonstrated the feasibility and efficacy of O₂ Nanosensor analysis in mouse and zebrafish muscle, providing support to test human MM subjects. They will validate a novel O₂ nanosensor-needle microarray device to quantify in vivo muscle OXPHOS function in adult MM human subjects.

All of the methods disclosed and claimed herein can be made and executed without undue experimentation in light of the present disclosure. While the compositions and methods of this disclosure have been described in terms of particular embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Lieberman, Pharmaceutical Dosage Forms (vols. 1-3, 1992)
Lloyd, The Art, Science and Technology of Pharmaceutical Compounding (1999) Pickar, Dosage Calculations, 1999.
Remington: The Science and Practice of Pharmacy, 20th Edition, 2003, Gennaro, Ed., Lippincott, Williams & Wilkins.
1. Gorman, G. S., et al., Prevalence of nuclear and mitochondrial DNA mutations related to adult mitochondrial disease. Ann Neurol, 2015. 77(5): p. 753-9.
2. Zolkipli-Cunningham, Z., et al., Mitochondrial disease patient motivations and barriers to participate in clinical trials. PLoS One, 2018. 13(5): p. e0197513.
3. Stacpoole, P. W., Why are there no proven therapies for genetic mitochondrial diseases? Mitochondrion, 2011. 11(5): p. 679-85.
4. Pfeffer, G., et al., Treatment for mitochondrial disorders. Cochrane Database Syst Rev, 2012(4): p. CD004426.
5. Koene, S., et al., Towards the harmonization of outcome measures in children with mitochondrial disorders. Dev Med Child Neurol, 2013. 55(8): p. 698-706.
6. Koene, S., et al., Developing outcome measures for pediatric mitochondrial disorders: which complaints and limitations are most burdensome to patients and their parents? Mitochondrion, 2013. 13(1): p. 15-24.
7. Koopman, W. J., et al., Mitochondrial disorders in children: toward development of small-molecule treatment strategies. EMBO Mol Med, 2016. 8(4): p. 311-27.
8. Schaefer, A. M., et al., Mitochondrial disease in adults: a scale to monitor progression and treatment. Neurology, 2006. 66(12): p. 1932-4.
9. Riazi, A., et al., Coordinating outcomes measurement in ataxia research: do some widely used generic rating scales tick the boxes? Mov Disord, 2006. 21(9): p. 1396-403.
10. Chinnery, P. F., Mitochondrial disease in adults: what's old and what's new? EMBO Mol Med, 2015. 7(12): p. 1503-12.
11. Frazier, A. E. and D. R. Thorburn, Biochemical analyses of the electron transport chain complexes by spectrophotometry. Methods Mol Biol, 2012. 837: p. 49-62.
12. Parikh, S., et al., Diagnosis and management of mitochondrial disease: a consensus statement from the Mitochondrial Medicine Society. Genet Med, 2015. 17(9): p. 689-701.
13. McCormick, E., E. Place, and M. J. Falk, Molecular genetic testing for mitochondrial disease: from one generation to the next. Neurotherapeutics, 2013. 10(2): p. 251-61.
14. Bank, W. and B. Chance, Diagnosis of defects in oxidative muscle metabolism by non-invasive tissue oximetry. Mol Cell Biochem, 1997. 174(1-2): p. 7-10.
15. Ozawa, T., et al., Extensive tissue oxygenation associated with mitochondrial DNA mutations. Biochem Biophys Res Commun, 1995. 213(2): p. 432-8.
16. Su Jin Lee, H. S. Y., Xing Xuan, JAe Park, Seung-Joon Paik, Mark Allen, A patch type non-enzymatic biosensor based on 3D SUS micro-needle electrode array for minimally invasive continuous glucose monitoring. Sensors and Actuators B 2016. 222: p. 1144-1151.
17. Reinke, C., et al., Effects of different acute hypoxic regimens on tissue oxygen profiles and metabolic outcomes. J Appl Physiol (1985), 2011. 111(3): p. 881-90.
18. Gorman, G. S., et al., Perceived fatigue is highly prevalent and debilitating in patients with mitochondrial disease. Neuromuscul Disord, 2015. 25(7): p. 563-6.
19. Taivassalo, T., et al., The spectrum of exercise tolerance in mitochondrial myopathies: a study of 40 patients. Brain, 2003. 126(Pt 2): p. 413-23.
20. Tarnopolsky, M., Exercise testing in metabolic myopathies. Phys Med Rehabil Clin N Am, 2012. 23(1): p. 173-86, xii.
21. Montes, J., et al., Six-Minute Walk Test demonstrates motor fatigue in spinal muscular atrophy. Neurology, 2010. 74(10): p. 833-8.
22. Mancuso, M., et al., International Workshop: Outcome measures and clinical trial readiness in primary mitochondrial myopathies in children and adults. Consensus recommendations. 16-18 Nov. 2016, Rome, Italy. Neuromuscul Disord, 2017. 27(12): p. 1126-1137.
23. Yi, S. H., et al., Validity of pediatric balance scales in children with spastic cerebral palsy. Neuropediatrics, 2012. 43(6): p. 307-13.
24. Kaufmann, P., et al., Prospective cohort study of spinal muscular atrophy types 2 and 3. Neurology, 2012. 79(18): p. 1889-97.
25. Robinson, P. N., et al., The Human Phenotype Ontology: a tool for annotating and analyzing human hereditary disease. Am J Hum Genet, 2008. 83(5): p. 610-5.
26. Wen Shen, L. S., Kacy Cullen, Mark Allen, FABRICATION OF NEURAL MICROELECTRODE ARRAYS FROM EXTRACELLULAR MATRIX. Solid-State Sensors, Actuators and Microsystems Workshop, 2016.
27. O'Byrne, M. L., et al., Habitual exercise correlates with exercise performance in patients with conotruncal abnormalities. Pediatr Cardiol, 2013. 34(4): p. 853-60.
28. Wittlieb-Weber, C. A., et al., Elevated left ventricular outflow tract velocities on exercise stress echocardiography may be a normal physiologic response in healthy youth. J Am Soc Echocardiogr, 2013. 26(12): p. 1372-8.
29. Bernier, F. P., et al., Diagnostic criteria for respiratory chain disorders in adults and children. Neurology, 2002. 59(9): p. 1406-11.
30. Taivassalo, T., et al., Endurance training and detraining in mitochondrial myopathies due to single large-scale mtDNA deletions. Brain, 2006. 129(Pt 12): p. 3391-401.
31. Dirks, I., et al., Assisted 6-minute cycling test: An exploratory study in children. Muscle Nerve, 2016. 54(2): p. 232-8.

What is claimed is:

1. An implantable oxygen (O₂) nanosensor comprising:
(a) an O₂ sensor comprising:
(i) a working electrode;
(ii) a reference electrode;
(iii) a counter electrode;
(iv) an inner electrolyte cell; and
(v) an O₂-permeable membrane bounding at least one portion of the inner electrolyte cell,
and optionally
(b) nanofiber material surrounding the O₂ sensor.

2. The nanosenor of claim 1, wherein the O₂ sensor is a Clark-type O₂ sensor comprising:
(a) a working electrode;
(b) a reference electrode;
(c) a counter electrode;
(d) an inner electrolyte cell;
(e) reaction chamber; and
(f) an O₂-permeable membrane separating the inner electrolyte cell and the reaction chamber.

3. The nanosensor of claim 1, wherein the nanofiber material is in the form of a nanofiber mesh tube.

4. The nanosensor of claim 3, wherein the nanofiber mesh tube is a polycaprolactone nanofiber mesh tube.

5. The nanosensor of claim 1, further comprising a voltage source and/or an ammeter.

6. The nanosensor of claim 1, wherein said nanosensor is about 2.5 mm in diameter, about 1.8 mm in diameter, or 1.8 mm to 1.0 mm in diameter, or 2.5 mm to 1.0 mm in diameter, or less than 1.0 mm in diameter.

7. The nanosensor of claim 1, wherein the working electrode is a platinum electrode or a silver electrode.

8. The nanosensor of claim 1, wherein the nanosensor may comprise materials that are biodegradable, such as wires that are biodegradable.

9. The nanosensor of claim 1, wherein the O₂-permeable membrane is a Teflon membrane.

10. The nanosensor of claim 1, wherein the O₂-permeable membrane is a permselective membrane.

11. A method of measuring oxygen (O₂) level in a tissue in vivo comprising (i) implanting the implantable O₂ nanosensor of claim 1 into a muscle tissue of a subject, and (ii) assessing O₂ level as a function of the conversion of O₂ into H₂O and resulting current generated.

12. The method of claim 11, wherein the subject is a non-human animal.

13. The method of claim 11, wherein in the subject is a human.

14. The method of claim 11, wherein implanting is into the subjects forearm muscle or gluteal muscle.

15. The method of claim 11, wherein implanting comprises inserting said O₂ nanosensor into a needle or needle array, introducing said needle or needle array into said muscle tissue, and deploying said O₂ nanosensor into said muscle tissue.

16. The method of claim 11, wherein after step (i), and before step (ii), said subject is subjected to physical exercise.

17. The method of claim 16, wherein physical exercise is to mild.

18. The method of claim 16, wherein physical exercise is to exhaustion.

19. The method of claim 11, wherein said subject is suspected of or diagnosed as suffering from disease or disorder.

20. The method of claim 19, wherein said disease or disorder is a myopathy, such as mitochondrial myopathy.

21. The method of claim 11, wherein implanting is into the subjects brain, heart, liver, intestines, pancreas, urinary bladder, uterus, kidney, adrenal gland, thyroid gland, bone, cartilage, joint, or eye.

22. The method of claim 11, wherein said subject is acutely ill, such as but not exclusively from trauma, surgery, infection, sepsis, stroke, heart attack, hemorrhage or shock.

23. The method of claim 11, wherein said subject is being medically monitored for resuscitation purposes.

24. The method of claim 11, wherein said subject is being evaluated on the sports field or for medical purposes for post-concussion mitochondrial pathology.

25. The method of claim 11, wherein said subject is in the general population and seeking to optimize their exercise training regimen.

26. The method of claim 11, wherein said subject is in the general population and seeking to optimize their nutrition regimen.

27. The method of claim 11, wherein said subject is in the general population and seeking to evaluate mitochondrial effects of their medication.

28. The method of claim 11, wherein said subject is in the general population and seeking to evaluate mitochondrial effects of lifestyle choices, including but not limited to diet, medications, exercise, drug use, tobacco exposure, environmental toxin exposure, chemical exposure

29. The method of claim 11, wherein said subject is an athlete or participating in athletic training.

30. The method of claim 11, wherein said subject is a military recruit or member.

31. The method of claim 11, wherein said subject has a family member with mitochondrial disease or dysfunction.

32. The method of claim 31, wherein said disease or disorder is a primary or secondary mitochondrial disorder without known myopathic features.

33. The method of claim 31, wherein said disease or disorder may involve secondary mitochondrial dysfunction.

34. The method of claim 31, wherein said subject is being evaluated for disease prognosis or progression.

35. The method of claim 31, wherein said subject is being evaluated for therapeutic response to a candidate therapy, therapies, or therapeutic intervention.

36. The nanosensor of claim 1, where the sensor materials are biodegradable.

37. The nanosensor of claim 1, where the wires are biodegradable.

38. The nanosensor of claim 1, where the sensor is attached by wires to a detector for analysis.

39. The nanosensor of claim 1, where the sensor is remotely analyzed without wire attachment.

40. The nanosensor of claim 1, where the sensor is embedded in a transdermal puncture device.

41. The nanosensor of claim 1, where the device is inserted from an external puncture device or needle microarray.

42. The method of claim 11, where the purpose is for safety assessments of exercise performance, capacity, and training.

43. The method of claim 11, where the purpose is for safety assessments in post-concussion.

44. The method of claim 11, where the purpose is for safety assessments in military training or injuries.

45. The method of claim 11, where the purpose is for diagnosis of primary (genetic based) mitochondrial disease.

46. The method of claim 11, where the purpose is for diagnosis of secondary mitochondrial disease.

47. The method of claim 11, where the purpose is for monitoring in vivo mitochondrial respiratory capacity.

48. The method of claim 11, where the purpose is for predicting and assessing organ failure at a pre-critical or critical level.

49. The method of claim 11, where the purpose is as a clinical trial outcome measure to assess mitochondrial disease or dysfunction natural history and progression.

50. The method of claim 11, where the purpose is as a clinical trial outcome measure to assess mitochondrial disease response to a candidate therapy, therapies, or therapeutic intervention

51. The method of claim 11, where the purpose is as a clinical diagnostic test for mitochondrial dysfunction.

52. The method of claim 11, where the purpose is as a clinical diagnostic test for mitochondrial disease severity, progression, and therapeutic response.

53. The nanosensor of claim 1, where the sensor(s) detects O2 levels alone and/or in combination with one or more of the following physiologic read-outs including but not limited to: calcium, potassium, sodium, chloride, pH, bicarbonate, carbon dioxide, hydrogen peroxide, temperature, lactate, pyruvate, nicotinamide adenine dinucleotide (NADH or NAD+), adenosine triphosphate (ATP), adenosine diphosphate (ADP), adenosine monophosphate (AMP), ammonia, acetoacetate, beta hydroxybutyrate, or emitted light.
